(19) United States
(12) Patent Application Publication
(10) Pub. No.: US 2012/0258146 A1
(43) Pub. Date: Oct. 11, 2012

(54) Method of treating organic diseases of nervous system, pschoorganic syndrome and encephalopathy

(76) Inventor: Oleg Iliich Epshtein, Moscow (RU)

(21) Appl. No.: 13/135,896

(22) Filed: Jul. 15, 2011

(30) Foreign Application Priority Data
Jul. 21, 2010 (RU) 2010130355
Jul. 1, 2011 (RU) 2011127059

(51) Int. Cl. A61K 39/395; A61P 25/00; A61P 25/16; A61K 9/00

(52) U.S. Cl. 424/400; 424/158.1; 424/146.1

(57) ABSTRACT

The present invention relates to a method of treating organic disease of the nervous system, psychoorganic syndrome or encephalopathy of various genesis by administration of activated-potentiated form of antibodies to brain-specific protein S-100 and activated-potentiated form of antibodies to endothelial NO synthase.

### FIELD

The present invention relates to the treatment of organic diseases of nervous system, psychoorganic syndrome and encephalopathy of different origin by administration of activated-potentiated form of antibodies to protein S-100 and activated-potentiated form of antibodies to endothelial NO-synthase.

### BACKGROUND

Psychoorganic syndrome is characterized by loss of memomry, reduction of intellect and lack of emotional control (Walther-Buel's triad). Asthenic phenomena are often observed. Memory disorder to a greater or lesser extent is observed. Hypomnesis (weakened or abnormally poor memory) is present with a tendency for permanency, namely dysmnesia; amnesia and confabulations are also possible. Scope of attention is considerably reduced and distraction is increased. Orientation becomes worse at the beginning in the environment and then individually. Level of thinking is reduced, that is there is a development of the lessening of conceptions, weakness of judgments, inability to adequately estimate the situation and one's own possibilities. The tempo of understanding is slowed-down, torpidity of thinking is combined with inclination to detailing and perseverations. Psychoorganic syndrome (organic psychosyndrome) is the state of psychic weakness caused by organic cerebral affection (vascular diseases of brain, affection of central nervous system, in case of syphilis, craniocerebral traumas, various intoxications, chronic metabolic diseases, by tumors and cerebral abscesses, encephalitis and within diseases accompanied by convulsive attacks). But most often psychoorganic syndrome occurs by arthrophytic processes of brain within presenile and old age (Alzheimer's disease, dotage). In the minor form psychoorganic syndrome represents asthenic state with weakness, increased exhausting, emotional lability, instability of attention and reduction of efficiency. Within hard forms of psychoorganic syndrome there is first a reduction in cognition followed by dementia.

Organic diseases of nervous system are vascular diseases of central nervous system (consequences of stroke, discirculatory encephalopathy), degenerative diseases of CNS, demyelinating diseases of nervous system, hereditary diseases of CNS etc.

Parkinson's disease is a chronic, progressive neurodegenerative disease caused by loss of cells that contain dopamine. Degeneration of dopaminergic neurons results in disorder of dopamine synthesis and finally in expressed motor disturbances, disorders of coordination of movements and deterioration of patients' life.

Encephalopathy is the common name for non-inflammatory (in contrast to encephalitis) of cerebral diseases. Encephalopathy can be inborn and acquired (organic affections of brain connected with intoxications, infections, alcoholism, traumas, hypovitaminosis, vascular diseases of brain, lack of vitamin B1). Manifestations: mainly pseudoneurotic and psychopathy-like manifestations. Treatment of encephalopathy depends on cause that evoked it.

Stroke is acute disturbance of cerebral blood flow characterized by sudden (within some minutes, hours) appearance of focal and/or general cerebral neurologic symptomatology that is preserved for more than 24 hours and results in patient's death within the shortest interval of time as a result of cerebrovascular pathology. Strokes include cerebral infarction, cerebral hemorrhage and subarachnoid hemorrhage that have etiopathogenetic and clinical distinctions.

Treatment of psychoorganic syndrome through neurotropic drug based on activated-poteniative form of antibodies to brain specific protein S-100 (RU 2156621 C1, A61K39/395, 2000) is known in the art. However the efficiency of this medication in most cases is not very high for treating different organic diseases of nervous system, psychoorganic syndrome and encephalopathy of various genesis. Thus, there is a continuing need for new drug products with desired therapeutic efficacy for treatment of organic diseases of nervous system, psychoorganic syndrome and encephalopathy of various genesis.

The therapeutic effect of an extremely diluted form (or ultra-low form) of antibodies potentized by homeopathic technology (activated potentiated form) has been discovered by the inventor of the present patent application, Dr. Oleg I. Epshtein. U.S. Pat. No. 7,582,294 discloses a medicament for treating Benign Prostatic Hyperplasia or prostatitis by administration of a homeopathically activated form of antibodies to prostate specific antigen (PSA). U.S. Pat. No. 7,700,096 discloses a homeopathically potentized form of antibodies to endothelial NO-synthase.

The S-100 protein is a cytoplasmic acidic calcium binding protein found predominantly in the gray matter of the brain, primarily in glia and Schwann cells.

The protein exists in several homo- or heterodimeric isoforms consisting of two immunologically distinct subunits, alpha and beta. The S-100 protein has been suggested for use as an aid in the diagnosis and assessment of brain lesions and neurological damage due to brain injury, as in stroke. Yardan et al., Usefulness of S1008 Protein in Neurological Disorders, J Pak Med Assoc Vol. 61, No. 3, March 2011, which is incorporated herein by reference.

Ultra low doses of antibodies to S-100 protein have been shown to have anxiolytic, anti-asthenic, anti-aggressive, stress-protective, anti-hypoxic, anti-ischemic, neuroprotective and nootropic activity. See Castagne V. et al., Antibodies to S100 proteins have anxiolytic-like activity at ultra-low doses in the adult rat, J Pharm Pharmacol. 2008, 60(3):309-16; Epshtein 0.1, Antibodies to calcium-binding S100B protein block the conditioning of long-term sensitization in the terrestrial snail, Pharmacol Biochem Behay., 2009, 94(1):37-42; Voronina T. A. et al., Chapter 8. Antibodies to S-100 protein in anxiety-depressive disorders in experimental and clinical conditions. In “Animal models in biological psychiatry”, Ed. Kalueff A. V. N-Y, “Nova Science Publishers, Inc.”, 2006, pp. 137-152, all of which are incorporated herein by reference.

Nitric oxide (NO) is a gaseous molecule that has been shown to acts in the signaling of different biological processes. Endothelium-derived NO is a key molecule in regulation of vascular tone and its association with vascular disease has long been recognized. NO inhibits many processes known to be involved in the formation of atherosclerotic plaque, including monocyte adhesion, platelet aggregation and vascular smooth muscle cell proliferation. Another important role of endothelial NO is the protection of the vascular wall from the oxidative stress induced by its own metabolic products and by the oxidation products of lipids and lipoproteins. Endothelial dysfunction occurs at very early stages of atherosclerosis. It is therefore possible that deficiency in local NO availability could be a final common pathway that accelerates atherogenesis in humans. In addition to its role in the vascular endothelium, NO availability has been shown to modulate metabolism of lipoproteins. Negative correlation has been reported between plasma concentrations of NO metabolic products and plasma total and Low Density Lipoprotein [LDL] cholesterol levels while High Density Lipoprotein [HDL] improves vascular function in hypercholesterolaemic subjects. The loss of NO has considerable effect on the development of the disease. Diabetes mellitus is associated with increased rates of morbidity and mortality caused primarily by the accelerated development of atherosclerotic disease. Moreover, reports show that diabetics have impaired lung functions. It has been proposed that insulin resistance leads to airway inflammation. Habib et al., Nitric Oxide Measurement From Blood To Lungs, Is There A Link? Pak J Physiol 2007; 3(1).

Nitric oxide is synthesized by the endothelium from L-arginine by nitric oxide synthase (NO synthase). NO synthase occurs in different isoforms, including a constitutive form (cNOS) and an inducible form (iNOS). The constitutive form is present in normal endothelial cells, neurons and some other tissues.

### SUMMARY

The present invention provides a method of treating organic disease of nervous system, psychoorganic syndrome and encephalopathy, the method comprising administering a pharmaceutical composition comprising activated-potentiated form of antibodies to brain-specific protein S-100 and activated-potentiated form of antibodies to endothelial NO synthase as an additional strengthening component.

The present invention provides a method of treating Parkinson's disease, the method comprising administering a pharmaceutical composition comprising activated-potentiated form of antibodies to brain-specific protein S-100 and activated-potentiated form of antibodies to endothelial NO synthase as an additional strengthening component.

The present invention provides a method of treating acute disturbance of cerebral blood flow-stroke, the method comprising administering a pharmaceutical composition comprising activated-potentiated form of antibodies to brain-specific protein S-100 and activated-potentiated form of antibodies to endothelial NO synthase as an additional strengthening component.

In one variant, the present invention provides a combination pharmaceutical composition comprising activated-potentiated form of antibodies to brain-specific protein S-100 and activated-potentiated form of antibodies to endothelial NO synthase, wherein the antibody is to the entire protein S-100 or fragments thereof.

In one variant, the present invention provides a combination pharmaceutical composition comprising activated-potentiated form of antibodies to brain-specific protein S-100 and activated-potentiated form of antibodies to endothelial NO synthase, wherein the antibody is to the entire endothelial NO synthase or fragments thereof.

In one variant, the combination pharmaceutical composition of this aspect of the invention includes activated-potentiated form of an antibody to protein S-100 which is in the form of a mixture of (C12, C30, and C50) or (C12, C30 and C200) homeopathic dilutions impregnated onto a solid carrier. The activated-potentiated form of an antibody to NO synthase is in the form of mixture of (C12, C30, and C50) or (C12, C30 and C200) homeopathic dilutions may be subsequently impregnated onto the solid carrier.

In one variant, the combination pharmaceutical composition of this aspect of the invention includes activated-potentiated form of an antibody to endothelial NO synthase which is in the form of a mixture of (C12, C30, and C50) or (C12, C30 and C200) homeopathic dilutions impregnated onto a solid carrier. The activated-potentiated form of an antibody to protein S-100 is in the form of mixture of (C12, C30, and C50) or (C12, C30 and C200) homeopathic dilutions may be subsequently impregnated onto the solid carrier.

Preferably, the activated-potentiated form of an antibody to protein S-100 is a monoclonal, polyclonal or natural antibody, more preferably, a polyclonal antibody. In one variant of this aspect of the invention, the activated-potentiated form of an antibody to a protein S-100 is prepared by successive centesimal dilutions coupled with shaking of every dilution. Vertical shaking is specifically contemplated.

Preferably, the activated-potentiated form of an antibody to endothelial NO synthase is a monoclonal, polyclonal or natural antibody, more preferably, a polyclonal antibody. In one variant of this aspect of the invention, the activated-potentiated form of an antibody to NO synthase is prepared by successive centesimal dilutions coupled with shaking of every dilution. Vertical shaking is specifically contemplated

In one variant of the invention, there is provided administration of from one to two unit dosage forms of the activated-potentiated form of an antibody to protein S-100 and one to two unit dosage forms of the activated-potentiated form of an antibody to endothelial NO synthase, each of the dosage form being administered from once daily to six times daily. Preferably, the one to two unit dosage forms of each of the activated-potentiated forms of antibodies is administered twice daily.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies”.

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated’ form of the antibodies of the present invention is not attributable to the initial molecular form of the antibody. Preferred is the “activated-potentiated” form of antibody in liquid or solid form in which the concentration of the initial molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the initial molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

In one aspect, the present invention provides a combination pharmaceutical composition comprising a) an activated-potentiated form of an antibody to NO synthase and b) an activated-potentiated form of an antibody to brain-specific protein S-100. As set forth herein above, each of the individual components of the combination is generally known for its won individual medical uses. However, the inventors of the present application surprisingly discovered that administration of the combination remarkably is useful for the treatment of psychoorganic disorders and encephalopathy.

In another aspect, the invention provides the method of treatment of psychoorganic disorders and encephalopathy by means of insertion in an organism of activated-potentiated form of antibodies to brain-specific protein S-100 simultaneously with activated-potentiated form of antibodies to endothelial NO synthase in ultra-low doses of affinity purified antibodies.

Preferably, for the purpose of treatment, the combination pharmaceutical composition is administered from once daily to four times daily, each administration including one or two combination unit dosage forms.

The pharmaceutical composition of the present application for the purpose of treatment of psychoorganic disorders and encephalopathy contains active components in volume primarily in 1:1 ratio.

For the purpose of treatment of psychoorganic disorders and encephalopathy the components of the pharmaceutical composition may be administered separately. However, the simultaneous administration of the combined components in one form of solutions and/or solid dosage form (tablet), which contains activated-potentiated form of antibodies to brain-specific protein S-100 and, accordingly, activated-potentiated form of antibodies to endothelial NO synthase is preferred.

In addition, during treatment of psychoorganic disorders and encephalopathy, separate and simultaneous application (intake to organism) of the declared pharmaceutical composition in the form of two separately prepared medications both in the form of solutions and solid dosage forms (tablets) each of which contains activated-potentiated form of antibodies to endothelial NO-synthase or to S-100 protein is possible.

The medical product is prepared mainly as follows.

The combination pharmaceutical composition in accordance with the present invention may be in the liquid form or in solid form. Each of the activated potentiated forms of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsynan, 1987, p. 9-33; “Hum. Antibodies. Monoclonal and recombinant antibodies, 30 years after” by Laffly E., Sodoyer R.—2005—Vol. 14.-N 1-2. P.33-55, both incorporated herein by reference.

Monoclonal antibodies may be obtained, e.g., by means of hybridoma technology. The initial stage of the process includes immunization based on the principles already developed in course of polyclonal antisera preparation. Further stages of work involve production of hybrid cells generating clones of antibodies with identical specificity. Their separate isolation is performed using the same methods as in case of polyclonal antisera preparation.

Polyclonal antibodies may be obtained via active immunization of animals. For this purpose, for example, suitable animals (e.g. rabbits) receive a series of injections of the appropriate antigen: brain-specific protein S-100 and endothelial NO synthase. The animals' immune system generates corresponding antibodies, which are collected from the animals in a known manner. This procedure enables preparation of a monospecific antibody-rich serum.

If desired, the serum containing antibodies may be purified, e.g., using affine chromatography, fractionation by salt precipitation, or ion-exchange chromatography. The resulting purified, antibody-enriched serum may be used as a starting material for preparation of the activated-potentiated form of the antibodies. The preferred concentration of the resulting initial solution of antibody in the solvent, preferably, water or water-ethyl alcohol mixture, ranges from about 0.5 to about 5.0 mg/ml.

The preferred procedure for preparing each component is the use of the mixture of three aqueous-alcohol dilutions of the primary matrix solution of antibodies diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the combination of the invention is polyclonal antibodies to brain-specific protein S-100 and endothelial NO synthase an initial (matrix) solution with concentration of 0.5 to 5.0 mg/ml is used for the subsequent preparation of activated-potentiated forms.

To prepare the pharmaceutical composition preferably polyclonal antibodies to brain-specific protein S-100 and endothelial NO synthase are used.

Polyclonal antibodies to endothelial NO synthase are obtained using adjuvant as immunogen (antigen) for immunization of rabbits and whole molecule of bovine endothelial NO synthase of the following sequence:

```
SEQ ID NO: 1
Met Gly Asn Leu Lys Ser Val Gly Gln Glu Pro Gly Pro Pro Cys
  1 5 10 15
Gly Leu Gly Leu Gly Leu Gly Leu Gly Leu Cys Gly Lys Gln Gly
  16 20 25 30
Pro Ala Ser Pro Ala Pro Glu Pro Ser Arg Ala Pro Ala Pro Ala
  31 35 40 45
Thr Pro His Ala Pro Asp His Ser Pro Ala Pro Asn Ser Pro Thr
  46 50 55 60
Leu Thr Arg Pro Pro Glu Gly Pro Lys Phe Pro Arg Val Lys Asn
  61 65 70 75
Trp Glu Leu GLys er Ile Thr Tyr Asp Thr Leu Cys Ala Gln Ser
  76 80 85 90
Gln Gln Asp Gly Pro Cys Thr Pro Arg Cys Cys Leu GLys er Leu
91 95 100 105
Val Leu Pro Arg Lys Leu Gln Thr Arg Pro Ser Pro Gly Pro Pro
106 110 115 120
Pro Ala Glu Gln Leu Leu Ser Gln Ala Arg Asp Phe Ile Asn Gln
121 125 130 135
Tyr Tyr Ser Ser Ile Lys Arg Ser GLys er Gln Ala His Glu Glu
136 140 145 150
Arg Leu Gln Glu Val Glu Ala Glu Val Ala Ser Thr Gly Thr Tyr
151 155 160 165
His Leu Arg Glu Ser Glu Leu Val Phe Gly Ala Lys Gln Ala Trp
166 170 175 180
Arg Asn Ala Pro Arg Cys Val Gly Arg Ile Gln Trp Gly Lys Leu
181 185 190 195
Gln Val Phe Asp Ala Arg Asp Cys Ser Ser Ala Gln Glu Met Phe
196 200 205 210
Thr Tyr Ile Cys Asn His Ile Lys Tyr Ala Thr Asn Arg Gly Asn
211 215 220 225
Leu Arg Ser Ala Ile Thr Val Phe Pro Gln Arg Ala Pro Gly Arg
226 230 235 240
Gly Asp Phe Arg Ile Trp Asn Ser Gln Leu Val Arg Tyr Ala Gly
241 245 250 255
Tyr Arg Gln Gln Asp GLys er Val Arg Gly Asp Pro Ala Asn Val
256 260 265 270
Glu Ile Thr Glu Leu Cys Ile Gln His Gly Trp Thr Pro Gly Asn
271 275 280 285
Gly Arg Phe Asp Vai Leu Pro Leu Leu Leu Gln Ala Pro Asp Glu
286 290 295 300
Ala Pro Glu Leu Phe Val Leu Pro Pro Glu Leu Val Leu Glu Val
301 305 310 315
Pro Leu Glu His Pro Thr Leu Glu Trp Phe Ala Ala Leu Gly Leu
316 320 325 330
Arg Trp Tyr Ala Leu Pro Ala Val Ser Asn Met Leu Leu Glu Ile
331 335 340 345
Gly Gly Leu Glu Phe Ser Ala Ala Pro Phe Ser Gly Trp Tyr Met
346 350 355 360
Ser Thr Glu Ile Gly Thr Arg Asn Leu Cys Asp Pro His Arg Tyr
361 365 370 375
Asn Ile Leu Glu Asp Val Ala Val Cys Met Asp Leu Asp Thr Arg
376 380 385 390
Thr Thr Ser Ser Leu Trp Lys Asp Lys Ala Ala Val Glu Ile Asn
391 395 400 405
Leu Ala Val Leu His Ser Phe Gln Leu Ala Lys Val Thr Ile Val
406 410 415 420
Asp His His Ala Ala Thr Val Ser Phe Met Lys His Leu Asp Asn
421 425 430 435
Glu Gln Lys Ala Arg Gly Gly Cys Pro Ala Asp Trp Ala Trp Ile
436 440 445 450
Val Pro Pro Ile Ser GLys er Leu Thr Pro Val Phe His Gln Glu
451 455 460 465
Met Val Asn Tyr Ile Leu Ser Pro Ala Phe Arg Tyr Gln Pro Asp
466 470 475 480
Pro Trp Lys GLy Ser Ala Thr Lys Gly Ala Gly Ile Thr Arg Lys
481 485 490 495
Lys Thr Phe Lys Glu Val Ala Asn Ala Val Lys Ile Ser Ala Ser
496 500 505 510
Leu Met Gly Thr Leu Met Ala Lys Arg Val Lys Ala Thr Ile Leu
511 515 510 525
Tyr Ala Ser Glu Thr Gly Arg Ala Gln Ser Tyr Ala Gln Gln Leu
526 530 535 540
Gly Arg Leu Phe Arg Lys Ala Phe Asp Pro Arg Vai Leu Cys Met
541 545 550 555
Asp Glu Tyr Asp Val Val Ser Leu Glu His Glu Ala Leu Val Leu
556 560 565 570
Val Val Thr Ser Thr Phe Gly Asn Gly Asp Pro Pro Glu Asn Gly
571 575 580 585
Glu Ser Phe Ala Ala Ala Leu Met Glu Met Ser Gly Pro Tyr Asn
586 590 595 600
Ser Ser Pro Arg Pro Glu Gln His Lys Ser Tyr Lys Ile Arg Phe
601 605 610 615
Asn Ser Val Ser Cys Ser Asp Pro Leu Val Ser Ser Trp Arg Arg
616 620 625 630
Lys Arg Lys Glu Ser Ser Asn Thr Asp Ser Ala Gly Ala Leu Gly
631 635 640 645
Thr Leu Arg Phe Cys Val Phe Gly Leu GLy Ser Arg Ala Tyr Pro
646 650 655 660
His Phe Cys Ala Phe Ala Arg Ala Val Asp Thr Arg Leu Glu Glu
661 665 670 675
Leu Gly Gly Glu Arg Leu Leu Gln Leu Gly Gln Gly Asp Glu Leu
676 680 685 690
Cys Gly Gln Glu Glu Ala Phe Arg Gly Trp Ala Lys Ala Ala Phe
691 695 700 705
Gln Ala Ser Cys Glu Thr Phe Cys Val Gly Glu Glu Ala Lys Ala
706 710 715 720
Ala Ala Gln Asp Ile Phe Ser Pro Lys Arg Ser Trp Lys Arg Gln
721 725 730 735
Arg Tyr Arg Leu Ser Thr Gln Ala Glu Gly Leu Gln Leu Leu Pro
736 740 745 750
Gly Leu Ile His Val His Arg Arg Lys Met Phe Gln Ala Thr Val
751 755 760 765
Leu Ser Val Glu Asn Leu Gln Ser Ser Lys Ser Thr Arg Ala Thr
766 770 775 780
Ile Leu Val Arg Leu Asp Thr Ala Gly Gln Glu Gly Leu Gln Tyr
781 785 790 795
Gln Pro Gly Asp His Ile Gly Ile Cys Pro Pro Asn Arg Pro Gly
796 800 805 810
Leu Val Glu Ala Leu Leu Ser Arg Val Glu Asp Pro Pro Pro Pro
811 815 820 825
Thr Glu Ser Val Ala Val Glu Gln Leu Glu Lys GLys er Pro Gly
826 830 835 840
Gly Pro Pro Pro Ser Trp Val Arg Asp Pro Arg Leu Pro Pro Cys
841 845 850 855
Thr Leu Arg Gln Ala Leu Thr Phe Phe Leu Asp Ile Thr Ser Pro
856 860 865 870
Pro Ser Pro Arg Leu Leu Arg Leu Leu Ser Thr Leu Ala Glu Glu
871 875 880 885
Pro Ser Glu Gln Gln Glu Leu Glu Thr Leu Ser Gln Asp Pro Arg
886 890 895 900
Arg Tyr Glu Glu Trp Lys Trp Phe Arg Cys Pro Thr Leu Leu Glu
901 905 910 915
Val Leu Glu Gln Phe Pro Ser Val Ala Leu Pro Ala Pro Leu Leu
916 920 925 930
Leu Thr Gln Leu Pro Leu Leu Gln Pro Arg Tyr Tyr Ser Val Ser
931 935 940 945
Ser Ala Pro Asn Ala His Pro Gly Glu Val His Leu Thr Val Ala
946 950 955 960
Val Leu Ala Tyr Arg Thr Gln Asp Gly Leu Gly Pro Leu His Tyr
961 965 970 975
Gly Val Cys Ser Thr Trp Leu Ser Gln Leu Lys Thr Gly Asp Pro
976 980 985 990
Val Pro Cys Phe Ile Arg Gly Ala Pro Ser Phe Arg Leu Pro Pro
991 995 1000 1005
Asp Pro Tyr Val Pro Cys Ile Leu Val Gly Pro Gly Thr Gly Ile
1006 1010 1015 1020
Ala Pro Phe Arg Gly Phe Trp Gln Glu Arg Leu His Asp Ile Glu
1021 1025 1030 1035
Ser Lys Gly Leu Gln Pro Ala Pro Met Thr Leu Val Phe Gly Cys
1036 1140 1145 1050
Arg Cys Ser Gln Leu Asp His Leu Tyr Arg Asp Glu Val Gln Asp
1051 1155 1160 1065
Ala Gln Glu Arg Gly Val Phe Gly Arg Val Leu Thr Ala Phe Ser
1066 1170 1175 1080
Arg Glu Pro Asp Ser Pro Lys Thr Tyr Val Gln Asp Ile Leu Arg
1081 1185 1190 1095
Thr Glu Leu Ala Ala Glu Val His Arg Val Leu Cys Leu Glu Arg
1096 1100 1105 1110
Gly His Met Phe Val Cys Gly Asp Val Thr Met Ala Thr Ser Val
1111 1115 1120 1125
Leu Gln Thr Val Gln Arg Ile Leu Ala Thr Glu Gly Asp Met Glu
1126 1130 1135 1140
Leu Asp Glu Ala Gly Asp Val Ile Gly Val Leu Arg Asp Gln Gln
1141 1145 1150 1155
Arg Tyr His Glu Asp Ile Phe Gly Leu Thr Leu Arg Thr Gln Glu
1156 1160 1165 1170
Val Thr Ser Arg Ile Arg Thr Gln Ser Phe Ser Leu Gln Glu Arg
1171 1175 1180 1185
His Leu Arg Gly Ala Val Pro Trp Ala Phe Asp Pro Pro Gly Pro
1186 1190 1195 1200
Asp Thr Pro Gly Pro
1201 1205
```

Polyclonal antibodies to NO synthase may be obtained using the whole molecule of human endothelial NO synthase of the following sequence:

```
SEQ ID NO: 2
Met Gly Asn Leu Lys Ser Val Ala Gln Glu Pro Gly Pro Pro Cys
1 5 10 15
Gly Leu Gly Leu Gly Leu Gly Leu Gly Leu Cys Gly Lys Gln Gly
  16 20 25 30
Pro Ala Thr Pro Ala Pro Glu Pro Ser Arg Ala Pro Ala Ser Leu
  31 35 40 45
Leu Pro Pro Ala Pro Glu His Ser Pro Pro Ser Ser Pro Leu Thr
  46 50 55 60
Gln Pro Pro Glu Gly Pro Lys Phe Pro Arg Val Lys Asn Trp Glu
  61 65 70 75
Val GLys er Ile Thr Tyr Asp Thr Leu Ser Ala Gln Ala Gln Gln
  76 80 85 90
Asp Gly Pro Cys Thr Pro Arg Arg Cys Leu GLys er Leu Val Phe
  91 95 100 105
Pro Arg Lys Leu Gln Gly Arg Pro Ser Pro Gly Pro Pro Ala Pro
106 110 115 120
Glu Gln Leu Leu Ser Gln Ala Arg Asp Phe Ile Asn Gln Tyr Tyr
121 125 130 135
Ser Ser Ile Lys Arg Ser GLys er Gln Ala His Glu Gln Arg Leu
136 140 145 150
Gln Glu Val Glu Ala Glu Val Ala Ala Thr Gly Thr Tyr Gln Leu
151 155 160 165
Arg Glu Ser Glu Leu Val Phe Gly Ala Lys Gln Ala Trp Arg Asn
166 170 175 180
Ala Pro Arg Cys Val Gly Arg Ile Gln Trp Gly Lys Leu Gln Val
181 185 190 195
Phe Asp Ala Arg Asp Cys Arg Ser Ala Gln Glu Met Phe Thr Tyr
196 200 205 210
Ile Cys Asn His Ile Lys Tyr Ala Thr Asn Arg Gly Asn Leu Arg
211 215 220 225
Ser Ala Ile Thr Val Phe Pro Gln Arg Cys Pro Gly Arg Gly Asp
226 230 235 240
Phe Arg Ile Trp Asn Ser Gln Leu Val Arg Tyr Ala Gly Tyr Arg
241 245 250 255
Gln Gln Asp GLy Ser Val Arg Gly Asp Pro Ala Asn Val Glu Ile
256 260 265 270
Thr Glu Leu Cys Ile Gln His Gly Trp Thr Pro Gly Asn Gly Arg
271 275 280 285
Phe Asp Val Leu Pro Leu Leu Leu Gln Ala Pro Asp Glu Pro Pro
286 290 295 300
Glu Leu Phe Leu Leu Pro Pro Glu Leu Val Leu Glu Val Pro Leu
301 305 310 315
Glu His Pro Thr Leu Glu Trp Phe Ala Ala Leu Gly Leu Arg Trp
316 320 325 330
Tyr Ala Leu Pro Ala Val Ser Asn Met Leu Leu Glu Ile Gly Gly
331 335 340 345
Leu Glu Phe Pro Ala Ala Pro Phe Ser Gly Trp Tyr Met Ser Thr
346 350 355 360
Glu Ile Gly Thr Arg Asn Leu Cys Asp Pro His Arg Tyr Asn Ile
361 365 370 375
Leu Glu Asp Val Ala Val Cys Met Asp Leu Asp Thr Arg Thr Thr
376 380 385 390
Ser Ser Leu Trp Lys Asp Lys Ala Ala Val Glu Ile Asn Val Ala
391 395 400 405
Val Leu His Ser Tyr Gln Leu Ala Lys Val Thr Ile Val Asp His
406 410 415 420
His Ala Ala Thr Ala Ser Phe Met Lys His Leu Glu Asn Glu Gln
421 425 430 435
Lys Ala Arg Gly Gly Cys Pro Ala Asp Trp Ala Trp Ile Val Pro
436 440 445 450
Pro Ile Ser GLys er Leu Thr Pro Val Phe His Gln Glu Met Val
451 455 460 465
Asn Tyr Phe Leu Ser Pro Ala Phe Arg Tyr Gln Pro Asp Pro Trp
466 470 475 480
Lys Gly Ser Ala Ala Lys Gly Thr Gly Ile Thr Arg Lys Lys Thr
481 485 490 495
Phe Lys Glu Val Ala Asn Ala Val Lys Ile Ser Ala Ser Leu Met
496 500 505 510
Gly Thr Val Met Ala Lys Arg Val Lys Ala Thr Ile Leu Tyr Gly
511 515 510 525
Ser Glu Thr Gly Arg Ala Gln Ser Tyr Ala Gln Gln Leu Gly Arg
526 530 535 540
Leu Phe Arg Lys Ala Phe Asp Pro Arg Val Leu Cys Met Asp Glu
541 545 550 555
Tyr Asp Val Val Ser Leu Glu His Glu Thr Leu Val Leu Val Val
556 560 565 570
Thr Ser Thr Phe Gly Asn Gly Asp Pro Pro Glu Asn Gly Glu Ser
571 575 580 585
Phe Ala Ala Ala Leu Met Glu Met Ser Gly Pro Tyr Asn Ser Ser
586 590 595 600
Pro Arg Pro Glu Gln His Lys Ser Tyr Lys Ile Arg Phe Asn Ser
601 605 610 615
Ile Ser Cys Ser Asp Pro Leu Val Ser Ser Trp Arg Arg Lys Arg
616 620 625 630
Lys Glu Ser Ser Asn Thr Asp Ser Ala Gly Ala Leu Gly Thr Leu
631 635 640 645
Arg Phe Cys Val Phe Gly Leu GLys er Arg Ala Tyr Pro His Phe
646 650 655 660
Cys Ala Phe Ala Arg Ala Val Asp Thr Arg Leu Glu Glu Leu Gly
661 665 670 675
Gly Glu Arg Leu Leu Gln Leu Gly Gln Gly Asp Glu Leu Cys Gly
676 680 685 690
Gln Glu Glu Ala Phe Arg Gly Trp Ala Gln Ala Ala Phe Gln Ala
691 695 700 705
Ala Cys Glu Thr Phe Cys Val Gly Glu Asp Ala Lys Ala Ala Ala
706 710 715 720
Arg Asp Ile Phe Ser Pro Lys Arg Ser Trp Lys Arg Gln Arg Tyr
721 725 730 735
Arg Leu Ser Ala Gln Ala Glu Gly Leu Gln Leu Leu Pro Gly Leu
736 740 745 750
Ile His Val His Arg Arg Lys Met Phe Gln Ala Thr Ile Arg Ser
751 755 760 765
Val Glu Asn Leu Gln Ser Ser Lys Ser Thr Arg Ala Thr Ile Leu
766 770 775 780
Val Arg Leu Asp Thr Gly Gly Gln Glu Gly Leu Gln Tyr Gln Pro
781 785 790 795
Gly Asp His Ile Gly Val Cys Pro Pro Asn Arg Pro Gly Leu Val
796 800 805 810
Glu Ala Leu Leu Ser Arg Val Glu Asp Pro Pro Ala Pro Thr Glu
811 815 820 825
Pro Val Ala Val Glu Gln Leu Glu Lys Gly Ser Pro Gly Gly Pro
826 830 835 840
Pro Pro Gly Trp Val Arg Asp Pro Arg Leu Pro Pro Cys Thr Leu
841 845 850 855
Arg Gln Ala Leu Thr Phe Phe Leu Asp Ile Thr Ser Pro Pro Ser
856 860 865 870
Pro Gln Leu Leu Arg Leu Leu Ser Thr Leu Ala Glu Glu Pro Arg
871 875 880 885
Glu Gln Gln Glu Leu Glu Ala Leu Ser Gln Asp Pro Arg Arg Tyr
886 890 895 900
Glu Glu Trp Lys Trp Phe Arg Cys Pro Thr Leu Leu Glu Val Leu
901 905 910 915
Glu Gln Phe Pro Ser Val Ala Leu Pro Ala Pro Leu Leu Leu Thr
916 920 925 930
Gln Leu Pro Leu Leu Gln Pro Arg Tyr Tyr Ser Val Ser Ser Ala
931 935 940 945
Pro Ser Thr His Pro Gly Glu Ile His Leu Thr Val Ala Val Leu
946 950 955 960
Ala Tyr Arg Thr Gln Asp Gly Leu Gly Pro Leu His Tyr Gly Val
961 965 970 975
Cys Ser Thr Trp Leu Ser Gln Leu Lys Pro Gly Asp Pro Val Pro
976 980 985 990
Cys Phe Ile Arg Gly Ala Pro Ser Phe Arg Leu Pro Pro Asp Pro
991 995 1000 1005
Ser Leu Pro Cys Ile Leu Val Gly Pro Gly Thr Gly Ile Ala Pro
1006 1010 1015 1020
Phe Arg Gly Phe Trp Gln Glu Arg Leu His Asp Ile Glu Ser Lys
1021 1025 1030 1035
Gly Leu Gln Pro Thr Pro Met Thr Leu Val Phe Gly Cys Arg Cys
1036 1040 1045 1050
Ser Gln Leu Asp His Leu Tyr Arg Asp Glu Val Gln Asn Ala Gln
1051 1055 1060 1065
Gln Arg Gly Val Phe Gly Arg Val Leu Thr Ala Phe Ser Arg Glu
1066 1070 1075 1080
Pro Asp Asn Pro Lys Thr Tyr Val Gln Asp Ile Leu Arg Thr Glu
1081 1085 1090 1095
Leu Ala Ala Glu Val His Arg Val Leu Cys Leu Glu Arg Gly His
1096 1100 1105 1110
Met Phe Val Cys Gly Asp Val Thr Met Ala Thr Asn Val Leu Gln
1111 1115 1120 1125
Thr Val Gln Arg Ile Leu Ala Thr Glu Gly Asp Met Glu Leu Asp
1126 1130 1135 1140
Glu Ala Gly Asp Val Ile Gly Val Leu Arg Asp Gln Gln Arg Tyr
1141 1145 1150 1155
His Glu Asp Ile Phe Gly Leu Thr Leu Arg Thr Gln Glu Val Thr
1156 1160 1165 1170
Ser Arg Ile Arg Thr Gln Ser Phe Ser Leu Gln Glu Arg Gln Leu
1171 1175 1180 1185
Arg Gly Ala Val Pro Trp Ala Phe Asp Pro Pro Gly Ser Asp Thr
1186 1190 1195 1200
Asn Ser Pro
1201 1203
```

To obtain polyclonal antibodies to NO synthase, it is also possible to use a fragment of endothelial NO synthase, selected, for example, from the following sequences:

```
SEQ ID NO: 3
Pro Trp Ala Phe
1192 1195
SEQ ID NO: 4
Gly Ala Val Pro
1189 1192
SEQ ID NO: 5
                                                                                                                Arg
                                                                                                              1185
His Leu Arg Gly Ala Val Pro Trp Ala Phe Asp Pro Pro Gly Pro
1186 1190 1195 1200
Asp Thr Pro Gly Pro
1201 1205
SEQ ID NO: 6
                                                                Ala Phe Asp Pro Pro Gly Pro
                                                                11941195 1200
Asp Thr Pro Gly Pro
1201 1205
SEQ ID NO: 7
His Leu Arg Gly Ala Val Pro Trp Ala Phe Asp
1186 1190 11951196
SEQ ID NO: 8
His Leu Arg Gly Ala Val Pro Trp Ala Phe Asp Pro Pro Gly Pro
1186 1190 1195 1200
Asp Thr Pro Gly Pro
1201 1205
```

The exemplary procedure for preparation of starting polyclonal antibodies to NO synthase may be described as follows: 7-9 days before blood sampling 1-3 intravenous injections are made to the rabbits to increase the level of polyclonal antibodies in the rabbit blood stream. Upon immunization, blood samples are taken to test the antibody level. Typically, the maximum level of the immune reaction of the soluble antigen is reached in 40-60 days after the first injection. After the termination of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube Product clots formed on the tube sides are removed with a wooden spatula, and a rod is placed into the clot in the tube center. The blood is then placed in a refrigerator for one night at the temperature of about 4° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. (or without addition NaN₃— at temperature −70° C.). To separate the target antibodies to endothelial NO synthase from the antiserum, the following solid phase absorption sequence is suitable:

(a) 10 ml of antiserum of rabbit is diluted twofold with 0.15 M NaCl, after which 6.26 g Na₂SO₄, is added, mixed and incubated for about 12-16 hours at 4° C.;

(b) the sediment is removed by centrifugation, dissolved in 10 ml of phosphate buffer and dialyzed against the same buffer within one night at room temperature;

(c) after the sediment is removed by centrifugation, the solution is put on the column with DEAE-cellulose, counterbalanced by phosphate buffer;

(d) the antibody fraction is determined by measuring the optical density of eluate at 280 nanometers.

The isolated crude antibodies are purified using affine chromatography method by attaching the obtained antibodies to endothelial NO synthase located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to endothelial NO synthase is 0.5 to 5.0 mg/ml, preferably, 2.0 to 3.0 mg/ml.

The brain-specific S100 protein, expressed by neurons and glial cells (astrocytes and oligodendrocytes), directly or through interactions with other proteins executes in the CNS a number of functions directed at maintaining normal brain functioning, including affecting learning and memory processes, growth and viability of neurons, regulation of metabolic processes in neuronal tissues and others. To obtain polyclonal antibodies to brain-specific protein S-100, brain-specific protein S-100 is used, which physical and chemical properties are described in the article of M. V. Starostin, S. M. Sviridov, Neurospecific Protein S-100, Progress of Modern Biology, 1977, Vol. 5, P. 170-178; found in the book M. B. Shtark, Brain-Specific Protein Antigenes and Functions of Neuron, “Medicine”, 1985; P. 12-14. Brain-specific protein S-100 is allocated from brain tissue of the bull by the following technique:
the bull brain tissue frozen in liquid nitrogen is converted into powder using a specialized mill;
proteins are extracted in the ratio of 1:3 (weight/volume) using an extracting buffer with homogenization;
the homogenate is heated for 10 min at 60° C. and then cooled to 4° C. in an ice bath;
thermolabile proteins are removed by centrifugation;
ammonium sulfate fractionation is carried out in stages, with subsequent removal of precipitated proteins;
the fraction containing S-100 protein is precipitated using 100% saturated ammonium sulfate accomplished by pH drop to 4.0; the desired fraction is collected by centrifugation;
the precipitate is dissolved in a minimum buffer volume containing EDTA and mercaptoethanol, the precipitate is dialyzed with deionized water and lyophilized;
fractionation of acidic proteins is followed by chromatography in ion-exchanging media, DEAE-cellulose DE-52 and then DEAE-sephadex A-50;
the collected and dialyzed fractions, which contain S-100 protein, are divided according to molecular weight by gel filtration on sephadex G-100;
purified S-100 protein is dialyzed and lyophilized.

The molecular weight of the purified brain-specific protein S-100 is 21000 D.

Owing to the high concentration of asparaginic and glutaminic acids brain-specific protein S-100 is highly acidic and occupies extreme anode position during electroendosmosis in a discontinuous buffer system of polyacrylamide gel which facilitates its identification.

The polyclonal antibodies to S-100 protein may also be obtained by a similar methodology to the methodology described for endothelial NO synthase antibodies using an adjuvant. The entire molecule of S-100 protein may be used as immunogen (antigen) for rabbits' immunization:

```
Bovine S100B
(SEQ ID NO: 9)
Met Ser Glu Leu Glu Lys Ala Val Val Ala Leu Ile Asp Val Phe
  1 5 10 15
His Gln Tyr Ser Gly Arg Glu Gly Asp Lys His Lys Leu Lys Lys
  16 20 25 30
Ser Glu Leu Lys Glu Leu Ile Asn Asn Glu Leu Ser His Phe Leu
  31 35 40 45
Glu Glu Ile Lys Glu Gln Glu Val Val Asp Lys Val Met Glu Thr
  46 50 55 60
Leu Asp Ser Asp Gly Asp Gly Glu Cys Asp Phe Gln Glu Phe Met
  61 65 70 75
Ala Phe Val Ala Met Ile Thr Thr Ala Cys His Glu Phe Phe Glu
  76 80 85 90
His Glu
  91 92
Human S100B
(SEQ ID NO: 10)
Met Ser Glu Leu Glu Lys Ala Met Val Ala Leu Ile Asp Val Phe
  1 5 10 15
His Gln Tyr Ser Gly Arg Glu Gly Asp Lys His Lys Leu Lys Lys
  16 20 25 30
Ser Glu Leu Lys Glu Leu Ile Asn Asn Glu Leu Ser His Phe Leu
  31 35 40 45
Glu Glu Ile Lys Glu Gln Glu Val Val Asp Lys Val Met Glu Thr
  46 50 55 60
Leu Asp Asn Asp Gly Asp Gly Glu Cys Asp Phe Gln Glu Phe Met
  61 65 70 75
Ala Phe Val Ala Met Val Thr Thr Ala Cys His Glu Phe Phe Glu
  76 80 85 90
His Glu
91 92
Human S100A1
(SEQ ID NO: 11)
Met Gly Ser Glu Leu Glu Thr Ala Met Glu Thr Leu Ile Asn Val
  1 5 10 15
Phe His Ala His Ser Gly Lys Glu Gly Asp Lys Tyr Lys Leu Ser
  16 20 25 30
Lys Lys Glu Leu Lys Glu Leu Leu Gln Thr Glu Leu Ser Gly Phe
  31 35 40 45
Leu Asp Ala Gln Lys Asp Val Asp Ala Val Asp Lys Val Met Lys
  46 50 55 60
Glu Leu Asp Glu Asn Gly Asp Gly Glu Val Asp Phe Gln Glu Tyr
  61 65 70 75
Val Val Leu Val Ala Ala Leu Thr Val Ala Cys Asn Asn Phe Phe
  76 80 85 90
Trp Glu Asn Ser
91 94
Bovine S100A1
(SEQ ID NO: 12)
Met Gly Ser Glu Leu Glu Thr Ala Met Glu Thr Leu Ile Asn Val
  1 5 10 15
Phe His Ala His Ser Gly Lys Glu Gly Asp Lys Tyr Lys Leu Ser
  16 20 25 30
Lys Lys Glu Leu Lys Glu Leu Leu Gln Thr Glu Leu Ser Gly Phe
  31 35 40 45
Leu Asp Ala Gln Lys Asp Ala Asp Ala Val Asp Lys Val Met Lys
  46 50 55 60
Glu Leu Asp Glu Asn Gly Asp Gly Glu Val Asp Phe Gln Glu Tyr
  61 65 70 75
Val Val Leu Val Ala Ala Leu Thr Val Ala Cys Asn Asn Phe Phe
  76 80 85 90
Trp Glu Asn Ser
91 94
```

To obtain antiserum, brain-specific S-100 protein or the mixture of S-100 protein s (antigens) in complex with methylated bull seralbumin as the carrying agent with full Freund's adjuvant is prepared and added to allocated brain-specific protein S-100 which is injected subdermally to a laboratory animal—a rabbit into area of back in quantity of 1-2 ml. On 8th, 15th day repeated immunization is made. Blood sampling is made (for example, from a vein in the ear) on the 26th and the 28th day.

The obtained antiserum titre is 1:500-1:1000, forms single precipitin band with an extract of nervous tissue but does not react with extracts of heterological bodies and forms single precipitin peak both with pure protein S-100 and with the extract of nervous tissue indicating that the antiserum obtained is monospecific.

The activated potentiated form of each component of the combination may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution—attenuation M) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to brain-specific protein S-100 (or to endothelial NO—synthase) with the concentration of 2.5 mg/ml is diluted in 99 parts of neutral aqueous or aqueous-alcohol solvent (preferably, 15%-ethyl alcohol) and then vertically shaken many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to brain-specific protein S-100 with the concentration of 2.5 mg/ml in 99 parts of a neutral solvent in different containers, which is equivalent to the centesimal homeopathic dilution C12. Similar procedures with the relevant dilution factor are performed to obtain dilutions C30, C50 and C 200. The intermediate dilutions may be tested in a desired biological model to check activity. The preferred activated potentiated forms for both antibodies comprising the combination of the invention are a mixture of C12, C30, and C200 dilutions or C12, C30 and C50 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, C49 and C199 respectively), and then one part of each component is added in one container according to the mixture composition and mixed with the required quantity of the solvent (e.g. with 97 parts for centesimal dilution).

Thus, activated-potentiated form of antibodies to brain-specific protein S-100 in ultra low dose is obtained by extra attenuation of matrix solution, accordingly in 100¹², 100³⁰ and 100²⁰⁰ times, equal to centesimal C12, C30 and C200 solutions or 100¹², 100³⁰ and 100⁵⁰ times, equal to centesimal C12, C30 and C50 solutions prepared on homoeopathic technology.

Use of active substance in the form of mixture of other various solutions on homoeopathic technology, for example, decimal and/or centesimal, (C12, C30, C100; C12, C30, C50; D20, C30, C100 or D10, C30, M100 etc.) is possible. The efficiency is defined experimentally.

External processing in the course of potentiation and concentration reduction can also be carried out by means of ultrasound, of electromagnetic or any other physical influence accepted in the homeopathic art.

Preferably, the combination pharmaceutical composition of the invention may be in the form of a liquid or in the solid unit dosage form. The preferred liquid form of the pharmaceutical composition is a mixture, preferably, at a 1:1 ratio of the activated potentiated form of antibodies to endothelial NO synthase and the activated potentiated form of antibodies to protein S-100. The preferred liquid carrier is water or water-ethyl alcohol mixture.

The solid unit dosage form of the pharmaceutical composition of the invention may be prepared by using impregnating a solid, pharmaceutically acceptable carrier with the mixture of the activated potentiated form aqueous or aqueous-alcohol solutions of active components that are mixed, primarily in 1:1 ratio and used in liquid dosage form. Alternatively, the carrier may be impregnated consecutively with each requisite dilution. Both orders of impregnation are acceptable.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

The example of preparation of the solid unit dosage form is set forth below. To prepare the solid oral form, 100-300 μm granules of lactose are impregnated with aqueous or aqueous-alcoholic solutions of the activated-potentiated form of antibodies to endothelial NO synthase and the activated potentiated form of antibodies to protein S-100 in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Hüttlin Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch—XL 400 tablet press) to form 150 to 500 mg round pills, preferably, 300 mg. After tableting, 300 mg pills are obtained that are saturated with aqueous-alcohol solution (3.0-6.0 mg/pill) of the combination of the activated-potentiated form of antibodies. Each component of the combination used to impregnate the carrier is in the form of a mixture of centesimal homeopathic dilutions, preferably, C12, C30 and C200.

Preferably, 1-2 tablets of the claimed pharmaceutical composition are administered 2-4 times a day.

The claimed pharmaceutical composition as well as its components does not possess sedative and myorelaxant effect, does not cause addiction and habituation.

### EXAMPLES

### Example 1

Study of the effect of a complex preparation containing ultralow doses of polyclonal affinity purified rabbit antibodies to brain-specific protein S-100 (anti-S100) and endothelial NO-synthase (anti-eNOS), obtained by super-dilution of initial matrix solution (concentration: 2.5 mg/ml) (100¹², 100³⁰, 100²⁰⁰ times), equivalent to a blend of centesimal homeopathic dilutions C12, C30, C200 (ratio: 1:1) (“ULD anti-S100+anti-eNOS”), as well as its components: ultralow doses (ULD) of polyclonal affinity purified rabbit antibodies to brain-specific protein S-100, purified on antigen, obtained by super-dilution of initial matrix solution (100¹², 100³⁰, 100²⁰⁰ times, equivalent to a blend of centesimal homeopathic dilution C12, C30, C200 (“ULD anti-S100”) and ultralow doses (ULD) of polyclonal rabbit antibodies to endothelial NO-synthase, obtained by super-dilution of initial matrix solution (100¹², 100³⁰, 100²⁰⁰ times), equivalent to a blend of centesimal homeopathic dilution C12, C30, C200 (“ULD anti-eNOS”) on in vitro on binding of standard ligand [³H]pentazocine to human recombinant σ1 receptor was evaluated using radioligand method. Potentiated distilled water (blend of homeopathic dilutions C12+C30+C200) was used as test preparations control.

The sigma-1 (σ1) receptor is an intracellular receptor which is localized in the cells of central nervous system, the cells of the most of peripheral tissues and immune component cells. These receptors exhibit a unique ability to be translocated which is thought to be caused by many psychotropic medications. The dynamics of sigma-1 receptors is directly linked to various influences which are performed by preparations acting to the sigma-1 receptors. These effects include the regulation of activity channels, ecocytosis, signal transfering, remodeling of the plasma membrane (formation of rafts) and lipid transportation/metabolism, all of which can contribute to the plasticity of neurons in a brain. There is evidence that the sigma-1 receptors have a modulating effect on all the major neuromediator systems: noradrenergic, serotonergic, dopaminergic, cholinergic systems and NMDA-adjustable glutamate effects. Sigma-1 receptors play an important role in the pathophysiology of neurodegenerative diseases (e.g., Alzheimer's disease, Parkinson), psychiatric and affective disorders and stroke and they also take part in the processes of learning and memory. In this regard, the ability of drugs to influence the efficiency of interaction of ligands with sigma-1 receptor is indicative of the presence of neuroprotective, anti-ischemic, anxiolytic, antidepressant and anti astenic components in the spectrum of its pharmacological activity and permits the consideration these drugs as effective preparations particularly for the treatment of cerebrovascular diseases.

During the test (to measure total binding) 20 μl of the complex preparation of ULD anti-S100+anti-eNOS or 10 μl of ULD anti-S100 or 10 μl of ULD anti-NOS were transferred in the incubation medium. Thus, the quantity of ULD anti-S100+anti-eNOS transferred into the test well when testing the complex preparation was identical to that of ULD anti-S100 and ULD anti-NOS tested as monopreparations, which allows for a comparison of the efficiency of the preparation to its separate components. 20 μl and 10 μl of potentiated water were transferred in the incubation medium.

Further, 160 μl (about 200 μg of protein) of Jurkat cell line membranes homogenate (human leukemic T-lymphocyte line), and finally, 20 μl of tritium-labeled radioligand [³H]pentazocine (15 nm) were transferred.

In order to measure non-specific binding, 20 μl of non-labeled ligand-haloperidol (10 μM) were transferred in the incubation medium instead of the preparations or potentiated water.

Radioactivity was measured using a scintillometer (Topcount, Packard) and scintillation blend (Microscint 0, Packard) following the incubation within 120 minutes at 22° C. in 50 mM Tris-HCl buffer (pH=7.4) and filtration using fiberglass filters (GF/B, Packard). Specific binding (during the test or control) was calculated as a difference between total (during the test or control) and non-specific binding.

Results are represented as percentage of specific binding inhibition in control (distilled water was used as control) (Table 1).

TABLE 1

| | | | | | % of |
|---|---|---|---|---|---|
| | | | | | radioligand |
| | | % of radioligand specific | | | binding |
| | Quantity per | binding in control | | | inhibition in |
| Test group | test basin | 1^{st} test | 2^{nd} test | Average | control |
| ULD anti- | 20 μl | 48.4 | 35.5 | 42.0 | 58.0 |
| S100 + anti- | | | | | |
| eNOS | | | | | |
| ULD anti- | 10 μl | 67.3 | 63.1 | 65.2 | 34.8 |
| S100 | | | | | |
| ULD anti- | 10 μl | 147.5 | 161.1 | 154.3 | −54.3 |
| eNOS | | | | | |
| Potentiated | 20 μl | 98.1 | 75.8 | 86.9 | 13.1 |
| water | | | | | |
| Potentiated | 10 μl | 140.1 | 106.2 | 123.2 | −23.2 |
| water | | | | | |

Effect of the preparations and potentiated water on binding of standard ligand [³H]pentazocine to human recombinant σ 1 receptor
Note:
% of specific binding in control = (specific binding during the test/specific binding in control) * 100%;
% of specific binding inhibition in control = 100% − (specific binding during the test/specific binding in control) * 100%).

The results reflecting inhibition above 50% represents significant effects of the tested compounds; inhibition from 25% to 50% confirms mild to moderate effects; inhibition less than 25% is considered to be insignificant effect of the tested compound and is within background level.

Therefore, this test showed that the complex preparation of ULD anti-S100+anti-eNOS is more efficient than its separate components (ULD anti-S100 and ULD anti-eNOS) in inhibiting the binding of standard radioligand [³H]pentazocine to human recombinant σ1 receptor; ULD anti-S100, transferred into the test basin, namely 10 μl, inhibit the binding of standard radioligand [3H]pentazocine to human recombinant σ1 receptor, but the effect intensity is inferior to that of the complex preparation of ULD anti-S100+anti-eNOS; ULD anti-eNOS, transferred into the test basin, namely 10 μl, had no effect on the binding of standard radioligand [3H]pentazocine to human recombinant σ1 receptor; potentiated water, transferred into the test basin, namely 10 μl or 20 μl, had no effect on the binding of standard radioligand [3H]pentazocine to human recombinant σ1 receptor.

### Example 2

To study the properties of the combination pharmaceutical composition of the present application for the treatment of acute cerebrovascular disease, tablets with weight of 300 mg were used. The tablets were impregnated with pharmaceutical composition containing water-alcohol solutions (6 mg/tab.) of activated-potentiated forms of polyclonal affinity purified rabbit brain-specific proteins antibodies S-100 (anti-S100) and to endothelial NO-synthase (anti-eNOS) in ultra low doses (ULD) obtained by super dilution of initial solution (with concentration of 2.5 mg/ml) in 100¹², 100³⁰, 100²⁰⁰ times, of equivalent mixture of centesimal homeopathic dilutions C12, C30, C200 (“ULD anti-S100+anti-eNOS”).

Subjects diagnosed with acute cerebrovascular disease (ACVD) of ischemic type in the system of right internal carotid artery (ischemic stroke) were in the study.

The control group received standardized vascular-metabolic support including non-narcotic analgesics and nonsteroid anti-inflammatory drugs, antiaggregants, anticoagulants and neuroprotective drugs.

The study was open randomized comparative clinical trial evaluating the efficacy of combination pharmaceutical composition ULD anti-S100+anti-eNOS as an add-on to standardized complex vascular-metabolic support versus standardized complex vascular-metabolic support (VMS) alone in treatment of patients in early recovery period of ischemic stroke.

The study included 12 patients aged 66.66±9.3 years old (from 50 to 84 years old) diagnosed with acute cerebrovascular disease of ischemic type in the system of right internal carotid artery

Compliance of patients to following inclusion and exclusion criteria was checked:

Inclusion Criteria:
1. Patients with acute cerebrovascular disease of ischemic type in the system of right internal carotid artery, no later than 21 days after the stroke onset at visit 1, confirmed by medical history, neurological examination and medical records.
2. Patients receiving standardized complex vascular-metabolic support in accordance with the standards of medical care in Moscow city starting on the first day of stroke onset.
3. Patients who are right-handers confirmed study of sensorimotor laterality according to T. A. Dobrokhotova and N. N. Bragina.
4. Patients with etiology of ACVD defined as a combination of atherosclerosis and arterial hypertension.
5. The patients with left-sided hemiparesis with increased of muscle tone by spastic type at Visit 1.
6. Total score on Birgitta Lindmark Motor Assessment Scale (Lindmark's scale) at Visit 0≧345 points.
7. Total score on Birgitta Lindmark Motor Assessment Scale (Lindmark's scale) at Visit 9 from 345 to 404 points.
8. No need for immunomodulatory drugs prescription for the next 6 months.
9. Patients with a level of education sufficient to adequately communicate with the researcher and study coordinator.
10. Patients assessed by the researcher as reliable and ready to perform all scheduled clinical visits, tests and procedures stipulated in the protocol.
11. Patients having a valid home address.
12. Patient signed the Informed Consent.

Exclusion Criteria:
1. Any brain surgery in medical history.
2. Acute myocardial infarction.
3. Hemorrhagic stroke.
4. The diagnosis of psychosis, bipolar disorder or schizoaffective disorder in medical history.
5. Major depressive disorder according to criteria of depression module of international neuropsychiatric mini-interview (MINI).
6. Factors/conditions of medical or another character which in the opinion of the researcher may affect to the test results for patients in the study.
7. Answers “2A”, “2B”, “2C” or “3” in the section “I” of Beck Depression questionnaire (active suicidal ideation with some intent to act, without a specific plan, or active suicidal ideation with a specific plan and intent).
8. Autoimmune disease in medical history.
9. Acute damage of liver or severe cirrhosis (class C by Child-Pugh).
10. Non-corrected disorder of thyroid gland function.
11. Decompensated arterial hypertension in medical history.
12. Serious or decompensated cardiovascular disease, liver disease, kidney disease, metabolic, respiratory or hematological disease, symptomatic peripheral vascular disease or another medical or psychiatric condition which in the opinion of the researcher, may affect the patient's participation in the study or could lead to prolonged hospitalization or re-hospitalization during the study.
13. Diseases and conditions which in the opinion of researcher may prevent patient from the participation in the study.
14. The intake of the drug containing ULD anti-eNOS or the drug containing ULD anti-S100 before inclusion in the study.
15. The intake of antidepressants of any group including plant and homeopathic preparations.
16. The intake of anxiolytics of any group including plant and homeopathic preparations.
17. The intake of immunomodulators including plant and homeopathic preparations.
18. The treatment with systemic steroids within 1 month before Visit 0.
19. The participation in the study of the drug containing ULD anti-eNOS or the drug containing ULD anti-S100 if patients took at least one doze of preparation.
20. Participation in other clinical studies within 1 month before within 1 month before being enrolled in this study.
21. Pregnancy, breast feeding, impossibility to use an adequate contraception during the study period and within 1 month after the last intake of the studied drug.
22. The presence of allergy/intolerance of any component of drugs including lactose intolerance.
23. Patients taking narcotic drugs and neuroleptics, alcoholic dependence, psychiatric diseases in patients.
24. Patients are the staff of the center which directly related to the conducted study and/or are family members of the research center staffs which directly associated with the ongoing study. The “family members” are a husband (wife), parents, children, brothers (sisters).
25. Participation in the trial or presumable receiving of compensation or participation in the judicial process in the opinion of a researcher.

After the determination of patient conformity to inclusion and exclusion criteria the patients were randomized into two study groups: a group of patients receiving ULD anti-S100+anti-eNOS in combination with a standardized complex vascular-metabolic support (6 patients, women—33.33%, men—66.66%, mean age—65.33±6.71 years old), a group of patients receiving the standardized complex vascular-metabolic support (VMS) (6 patients, women—50% men—50%, mean age—68.0±11.88 years old).

During this study the five visits were carried out. Treatment phase lasted from Visit 1 to Visit 4 for 84±5 days on average. Visit 4 (Day 84±5) was the first endpoint of the study followed by a follow-up observation. Follow-up phase continued from Visit 4 to Visit 5 (Day 168±5 on average).

In the safety analysis the data of all patients participating in the study (n=12) was included. During the study good tolerance of the drug was recorded. No adverse events were registered. All patients of studied groups have completed the treatment according to the protocol; no early dropouts.

The effect of ULD anti-S100+anti-eNOS preparation on the main clinical signs and symptoms of ACVD (Lindmark's scale), anxiety-depressive disorders symptoms (Beck Depression questionnaire) as well as the on patient's cognitive functions (The Mini Mental State Examination (MMSE)) were assessed. An improvement was found in the key symptoms of ACVD such as statistically significant reduction of Lindmark's scale total score (from 383.5±7.81 to 412.33±7.47, p<0.001), decrease of severity of anxiety-depressive disorders (statistically significant decrease of Beck Depression questionnaire total score from 12.16±3.6 to 6.33±4.76, p<0.05) as well as the statistically significant improvement of cognitive functions (change of MMSE questionnaire total score from 26.0±3.69 to 29.66±0.52, p<0.05) by the end of treatment (Table 2).

At that in the control group the only a tendency to ameliorate the main clinical signs and symptoms of ACVD was recorded (increase in Lindmark's scale total score from 379.5±8.12 to 394.66±13.67, p<0.05.)

In the intergroup comparison the significant difference in the dynamics of the main clinical signs and symptoms of ACVD assessed by Lindmark's scale was shown (compared with the control p<0.02). Intergroup differences in the dynamics of anxiety and depressive disorders symptoms did not reach statistically significant values, which may be due to insufficient number of patients.

No further changes in the studied parameters were revealed during the follow-up period.

TABLE 2

| | | Beck | |
|---|---|---|---|
| | | Depression | |
| | Lindmark's scale | questionnaire | MMSE |
| ULD anti-S100 + | 383.5 ± 7.81 | 12.16 ± 3.6 | 26.0 ± 3.69 |
| anti-eNOS before | | | |
| treatment | | | |
| ULD anti-S100 + | 412.33 ± 7.47**# | 6.33 ± 4.76* | 29.66 ± 0.52* |
| anti-eNOS after | | | |
| treatment | | | |
| Vascular- | 379.5 ± 8.12 | 11.66 ± 3.67 | 27.83 ± 2.48 |
| metabolic support | | | |
| (VMS) alone | | | |
| before treatmant | | | |
| VMS after | 394.66 ± 13.67* | 10.16 ± 2.714 | 28.5 ± 2.07 |
| treatment | | | |

*p vs baseline <0.05;
**p vs baseline <0.001;
#p vs control <0.02

Thus, in the conducted clinical study a positive effect of combined pharmaceutical composition ULD anti-S100+anti-eNOS is shown in combination with standardized complex vascular-metabolic support (VMS) on the main clinical signs and symptoms of ACVD and tendency to effect anxiety and depressive disorders symptoms and cognitive functions of the patients in early recovery period of ischemic stroke. In addition, good drug tolerability was confirmed. No drug-related adverse events were registered.

### Example 3

To study the properties of the combination pharmaceutical composition of the present application for the treatment of Parkinson disease, tablets with weight of 300 mg were used. The tablets were impregnated with pharmaceutical composition containing water-alcohol solutions (6 mg/tab.) of activated-potentiated forms of polyclonal affinity purified rabbit brain-specific proteins antibodies S-100 (anti-S100) and to endothelial NO-synthase (anti-eNOS) in ultra low doses (ULD) obtained by super dilution of initial solution (with concentration of 2.5 mg/ml) in 100¹², 100³⁰, 100²⁰⁰ times, of equivalent mixture of centesimal homeopathic dilutions C12, C30, C200 (“ULD anti-S100+anti-eNOS”).

The control group patients received 300 mg tablets impregnated with pharmaceutical composition containing water-alcohol solutions (6 mg/tablet) of activated-potentiated forms of polyclonal affinity purified rabbit brain-specific proteins antibodies S-100 (anti-S100) in ultra low doses (ULD) obtained by super dilution of initial solution (with concentration of 2.5 mg/ml) in 100¹², 100³⁰, 100²⁰⁰ times.

The study included patients with diagnosed Parkinson's disease. Parkinson's disease is characterized by progressive damage and destruction of dopamine neurons in the central nervous system.

The study was a single-center open-label randomized comparative clinical trial of efficiency and safety of the of ULD anti-S100+anti-eNOS ULD anti-S100, used as monotherapy, in combination with dopamine receptor agonists or in combination with levodopa in the treatment of patients with Parkinson's disease.

The study enrolled 14 patients aged 52-80 years old (67.93±11.56) with diagnosed Parkinson's disease (mean age was 67.93±11.56).

The study included the patients' conformity to the following inclusion and exclusion criteria:

Inclusion Criteria:
1. Patients with Parkinson disease, stage 1 to 5 by Hoehn and Yahr, confirmed by medical history (including positive levodopa test), neurological examinations and medical records.
2. Predominantly trembling, trembling-rigid or akinetico-rigid forms of Parkinson's disease.
3. Stages 1 to 2.5 by Hoehn and Yahr for patients receiving no anti-Parkinson's therapy at Visit 1.
4. Stages 1 to 5 by Hoehn and Yahr for patients receiving no anti-Parkinson's therapy at Visit 1.
5. No need for immunomodulatory drugs prescription for the next 6 months.
6. Patients with a level of education sufficient to adequately communicate with the researcher and study coordinator.
7. Patients assessed by the researcher as reliable and ready to perform all scheduled clinical visits, tests and procedures stipulated in the protocol.
8. Patients having a valid home address.
9. Patient signed the Informed Consent.

Exclusion Criteria:
1. Any brain surgery in medical history.
2. Acute myocardial infarction.
3. Hemorrhagic stroke.
4. The diagnosis of psychosis, bipolar disorder or schizoaffective disorder in medical history.
5. Major depressive disorder according to criteria of depression module of international neuropsychiatric mini-interview (MINI).
6. Factors/conditions of medical or another character which in the opinion of the researcher may affect to the test results for patients in the study.
7. Answers “2A”, “2B”, “2C” or “3” in the section “I” of Beck Depression questionnaire (active suicidal ideation with some intent to act, without a specific plan, or active suicidal ideation with a specific plan and intent).
8. Autoimmune disease in medical history.
9. Acute damage of liver or severe cirrhosis (class C by Child-Pugh).
10. Non-corrected disorder of thyroid gland function.
11. Decompensated arterial hypertension in medical history.
12. Serious or decompensated cardiovascular disease, liver disease, kidney disease, metabolic, respiratory or hematological disease, symptomatic peripheral vascular disease or another medical or psychiatric condition which in the opinion of the researcher, may affect the patient's participation in the study or could lead to prolonged hospitalization or re-hospitalization during the study.
13. Diseases and conditions which in the opinion of researcher may prevent patient from the participation in the study.
14. The intake of the drug containing ULD anti-eNOS or the drug containing ULD anti-S100 before inclusion in the study.
15. The intake of antidepressants of any group including plant and homeopathic preparations.
16. The intake of anxiolytics of any group including plant and homeopathic preparations.
17. The intake of immunomodulators including plant and homeopathic preparations.
18. The treatment with systemic steroids within 1 month before Visit 0.
19. The participation in the study of the drug containing ULD anti-eNOS or the drug containing ULD anti-S100 if patients took at least one doze of preparation.
20. Participation in other clinical studies within 1 month before within 1 month before being enrolled in this study.
21. Pregnancy, breast feeding, impossibility to use an adequate contraception during the study period and within 1 month after the last intake of the studied drug.
22. The presence of allergy/intolerance of any component of drugs including lactose intolerance.
23. Patients taking narcotic drugs and neuroleptics, alcoholic dependence, psychiatric diseases in patients.
24. Patients are the staff of the center which directly related to the conducted study and/or are family members of the research center staffs which directly associated with the ongoing study. The “family members” are a husband (wife), parents, children, brothers (sisters).
25. Participation in the trial or presumable receiving of compensation or participation in the judicial process in the opinion of a researcher.

After the patients's conformity to the inclusion and exclusion criteria had been checked, the patients were randomized into two study groups: group of patients receiving ULD anti-S100 in combination with anti-Parkinson's therapy (7 patients, women—57.14%, men—42.86%, mean age—65.25±10.6 years old), and group of patients receiving ULD anti-S100+anti-eNOS in combination with anti-Parkinson's therapy (7 people, women—57.14%, men—42.86%, mean age—71.5±12.79 years old).

During this study the five visits were carried out. Treatment phase lasted from Visit 1 to Visit 3 for 56±5 days on average. Visit 3 (Day 56±5) was the first endpoint of the study followed by a follow-up observation. Follow-up phase continued from Visit 3 to Visit 4 (Day 84±5 on average).

In the safety analysis the data of all patients participating in the study (n=124) was included. During the study good tolerance of the drug was recorded. No adverse events were registered. All patients of studied groups have completed the treatment according to the protocol; no early dropouts.

At the stage of designing the trial and selecting patients the study groups were divided into the following subgroups:
1. Patients at early stages of Parkinson's disease (1-2.5 by Hoehn and Yahr), receiving no anti-Parkinson's therapy, assigned to receive ULD anti-S100+anti-eNOS as monotherapy. The subgroup comprised 2 patients.
2. Patients at any stage of Parkinson's disease (1-5 by Hoehn and Yahr), receiving levodopa preparations without drug-related side effects specific for anti-Parkinson Disease's therapy, assigned to receive ULD anti-S100+anti-eNOS. The subgroup comprised 2 patients.
3. Patients at any stage of Parkinson's disease (1-5 by Hoehn and Yahr), receiving levodopa preparations with drug-related side effects specific for anti-Parkinson Disease's therapy (dyskinesia, on-off periods, hallucinations etc.), assigned to receive ULD anti-S100+anti-eNOS. The subgroup comprised 2 patients.
4. Patients at any stage of Parkinson's disease (1-5 by Hoehn and Yahr), receiving dopamine receptor agonists, assigned to receive ULD anti-S100+anti-eNOS. The subgroup comprised 1 patient.
5. Patients at early stages of Parkinson's disease (1-2.5 by Hoehn and Yahr), receiving no anti-Parkinson's therapy, assigned to receive ULD anti-S100. The subgroup comprised 1 patient.
6. Patients at any stage of Parkinson's disease (1-5 by Hoehn and Yahr), receiving levodopa preparations without drug-related side effects specific for anti-Parkinson Disease's therapy, assigned to receive ULD anti-S100. The subgroup comprised 2 patients.
7. Patients at any stage of Parkinson's disease (1-5 by Hoehn and Yahr), receiving levodopa preparations with drug-related side effects specific for anti-Parkinson Disease's therapy (dyskinesia, on-off periods, hallucinations etc.), assigned to receive ULD anti-S100. The subgroup comprised 2 patients.
8. Patients at any stage of Parkinson's disease (1-5 by Hoehn and Yahr), receiving dopamine receptor agonists, assigned to receive ULD anti-S100+anti-eNOS. The subgroup comprised 2 patients.

The study of effect of ULD anti-S100+anti-eNOS on the main clinical signs and symptoms of Parkinson's disease (UPDRS inventory), activity of the patient's everyday life (Schwab and England disability score), as well as the patient's anxious and depressive disorders (Beck depression inventory) revealed clinically significant effects of the therapy. Statistically significant reduction in the UPDRS total score (from 63.5±9.77 to 49.16±10.09, p<0.05) and increase in the activity of the patient's everyday life (statistically significant increase in the Schwab and England scale score (from 65.0±12.25 to 78.331:7.53, p<0.05) by Visit 3. A tendency for amelioration of anxious and depressive disorders was also found, which may be due to the small number of patients and within—group variation at the baseline (Table 3).

At that, a difference between the groups in the UPDRS total score by the end of therapy was statistically significant at p<0.05.

The same endpoints in the group of patients receiving ULD anti-S100, showed no trend for improvement, except a statistically significant decrease in the level of anxious and depressive disorders assessed by Beck depression inventory (from 12.0±7.08 to 6.0±2.52, p<0.05)

An analysis of the results of the study showed no statistically significant differences both between the subgroups receiving ULD anti-S100+anti-eNOS in combination with various therapy options at different stages of Parkinson's disease and between related subgroups receiving ULD anti-S100.

TABLE 3

| | | | Beck |
|---|---|---|---|
| | | Schwab and | depression |
| | UPDRS | England | inventory |
| ULD anti- | 63.5 ± 9.77 | 65.0 ± 12.25 | 18.28 ± 14.33 |
| S100 + anti-eNOS | | | |
| before treatment | | | |
| ULD anti- | 49.16 ± 10.09*# | 78.33 ± 7.53* | 8.67 ± 7.99 |
| S100 + anti-eNOS | | | |
| after treatment | | | |
| ULD anti-S100 | 70.125 ± 16.57 | 78.57 ± 13.45 | 12.0 ± 7.08 |
| before treatment | | | |
| ULD anti-S100 after | 66.75 ± 14.62 | 78.75 ± 12.46 | 6.0 ± 2.52* |
| treatment | | | |

*p vs baseline <0.05;
#p vs control <0.05

Thus, in the conducted clinical study a positive effect of combined pharmaceutical composition ULD anti-S100+anti-eNOS on the main clinical signs and symptoms of Parkinson's Disease and tendency to effect anxiety and depressive disorders symptoms. In addition, good drug tolerability was confirmed. No drug-related adverse events were registered.

### Example 4

To study the properties of the combination pharmaceutical composition of the present application for the treatment of psychoorganic disorders and encephalopathy, tablets with weight of 300 mg were used. The tablets were impregnated with pharmaceutical composition containing water-alcohol solutions (6 mg/tab.) of activated-potentiated forms of polyclonal affinity purified rabbit brain-specific proteins antibodies S-100 (anti-S100) and to endothelial NO-synthase (anti-eNOS) in ultra low doses (ULD) obtained by super dilution of initial solution (with concentration of 2.5 mg/ml) in 100¹², 100³⁰, 100²⁰⁰ times, of equivalent mixture of centesimal homeopathic dilutions C12, C30, C200 (“ULD anti-S100+anti-eNOS”).

The control group patients received 300 mg tablets impregnated with pharmaceutical composition containing water-alcohol solutions (6 mg/tablet) of activated-potentiated forms of polyclonal affinity purified rabbit brain-specific proteins antibodies S-100 (anti-S100) in ultra low doses (ULD) obtained by super dilution of initial solution (with concentration of 2.5 mg/ml) in 100¹², 100³⁰, 100²⁰⁰ times.

The study included patients diagnosed with psychoorganic syndrome of posttraumatic origin. Psychoorganic syndrome is characterized by the following triad of signs: weakness of memory, loop of intelligence, incontinence of affect (Walther Buel triad).

The study was an open-label randomized comparative parallel group clinical trial of efficacy and safety of the therapy in patients with psychoorganic syndrome of posttraumatic origin (the first group of patients took the preparation of ULD anti-S100, the second group of patients—the preparation of ULD anti-S100+anti-eNOS).

The study included 6 patients aged 35 to 90 years old (mean age 70.83±21.95) diagnosed with psychoorganic syndrome.

Compliance of patients to following inclusion and exclusion criteria was checked:

Inclusion Criteria:
1. Patients diagnosed with posttraumatic encephalopathy with psychoorganic syndrome or with encephalopathy of complex etiology (vascular, posttraumatic) with psychoorganic syndrome, confirmed by medical history, neurological examinations and medical records.
2. Patient without change in concomitant therapy within at least one month prior to Visit 1.
3. No need for change in concomitant therapy for the whole observation period.
4. No need for immunomodulatory drugs prescription for the next 6 months.
5. Patients with a level of education sufficient to adequately communicate with the researcher and study coordinator.
6. Patients assessed by the researcher as reliable and ready to perform all scheduled clinical visits, tests and procedures stipulated in the protocol.
7. Patients having a valid home address.

Exclusion Criteria:
1. Any brain surgery in medical history.
2. Acute myocardial infarction.
3. Hemorrhagic stroke.
4. The diagnosis of psychosis, bipolar disorder or schizoaffective disorder in medical history.
5. Major depressive disorder according to criteria of depression module of international neuropsychiatric mini-interview (MINI).
6. Factors/conditions of medical or another character which in the opinion of the researcher may affect to the test results for patients in the study.
7. Answers “2A”, “2B”, “2C” or “3” in the section “I” of Beck Depression questionnaire (active suicidal ideation with some intent to act, without a specific plan, or active suicidal ideation with a specific plan and intent).
8. Autoimmune disease in medical history.
9. Acute damage of liver or severe cirrhosis (class C by Child-Pugh).
10. Non-corrected disorder of thyroid gland function.
11. Decompensated arterial hypertension in medical history.
12. Serious or decompensated cardiovascular disease, liver disease, kidney disease, metabolic, respiratory or hematological disease, symptomatic peripheral vascular disease or another medical or psychiatric condition which in the opinion of the researcher, may affect the patient's participation in the study or could lead to prolonged hospitalization or re-hospitalization during the study.
13. Diseases and conditions which in the opinion of researcher may prevent patient from the participation in the study.
14. The intake of the drug containing ULD anti-eNOS or the drug containing ULD anti-S100 before inclusion in the study.
15. The intake of antidepressants of any group including plant and homeopathic preparations.
16. The intake of anxiolytics of any group including plant and homeopathic preparations.
17. The intake of immunomodulators including plant and homeopathic preparations.
18. The treatment with systemic steroids within 1 month before Visit 0.
19. The participation in the study of the drug containing ULD anti-eNOS or the drug containing ULD anti-S100 if patients took at least one doze of preparation.
20. Participation in other clinical studies within 1 month before within 1 month before being enrolled in this study.
21. Pregnancy, breast feeding, impossibility to use an adequate contraception during the study period and within 1 month after the last intake of the studied drug.
22. The presence of allergy/intolerance of any component of drugs including lactose intolerance.
23. Patients taking narcotic drugs and neuroleptics, alcoholic dependence, psychiatric diseases in patients.
24. Patients are the staff of the center which directly related to the conducted study and/or are family members of the research center staff's which directly associated with the ongoing study. The “family members” are a husband (wife), parents, children, brothers (sisters).
25. Participation in the trial or presumable receiving of compensation or participation in the judicial process in the opinion of a researcher.

After the determination of patient conformity to inclusion and exclusion criteria the patients were randomized into two study groups: a group of patients receiving ULD anti-S100 (3 patients, women—33.33%, men—66.66%, mean age—71.33±16.25 years old), a group of patients receiving ULD anti-S100+anti-eNOS (3 patients, women—66.66% men—33.33%, mean age—70.33±30.66 years old).

During this study the five visits were carried out. Treatment phase lasted from Visit 1 to Visit 4 for 84±5 days on average. Visit 4 (Day 84±5) was the first endpoint of the study followed by a follow-up observation. Follow-up phase continued from Visit 4 to Visit 5 (Day 168±5 on average).

In the safety analysis the data of all patients participating in the study (n=6) was included. During the study good tolerance of the drug was recorded. No adverse events were registered. All patients of studied groups have completed the treatment according to the protocol; no early dropouts.

The effect of ULD anti-S100+anti-eNOS preparation on the main clinical signs and symptoms of psychoorganic syndrome (NPI neuropsychiatric inventory, Intensity section), on the intensity of concomitant distress of the person attending to the patient (NPI Neuropsychiatric Inventory, Distress section) as well as the on patient's cognitive functions (The Mini Mental State Examination, MMSE) were assessed. An improvement was found in the key symptoms of psychoorganic syndrome such as statistically significant reduction of the intensity section of NPI neuropsychiatric inventory (from 91.0±15.13 to 69.0±+6.24, p<0.05), decrease of distress section score of NPI neuropsychiatric inventory (from 44.33±17.78 to 36.33±3.21, p<0.05) at Visit 4 (Table 4).

In the group of patients receiving ULD anti-S100 alone no clinical improvement was recorded.

At that, a difference between the groups of patients in the total score of the Intensity section of NPI neuropsychiatric inventory at the end of therapy was statistically significant at p<0.05.

TABLE 4

| | NPI | | | |
|---|---|---|---|---|
| | (intensity) | NPI (distress) | ADS-ADL | MMSE |
| ULD anti- | 91.0 + 15.13 | 44.33 + 17.78 | 42.66 + 4.93 | 22.33 + 3.21 |
| S100 + anti- | | | | |
| eNOS before | | | | |
| treatment | | | | |
| ULD anti- | 69.0 + 6.244*# | 36.33 + 3.21* | 52.0 + 5.57 | 22.66 + 2.08 |
| S100 + anti- | | | | |
| eNOS after | | | | |
| treatment | | | | |
| ULD anti-S100 | 114.0 + 25.53 | 45.66 + 14.47 | 33.0 + 13.89 | 22.33 + 4.16 |
| before | | | | |
| treatment | | | | |
| ULD anti-S100 | 99.66 + 18.0 | 49.0 + 17.05 | 31.66 + 10.69 | 23.0 + 4.36 |
| after treatment | | | | |

*p from baseline <0.05;
#p from control <0.05

Thus, in the conducted clinical study a positive effect of combined pharmaceutical composition ULD anti-S100+anti-eNOS on the main clinical signs and symptoms of psychoorganic syndrome and tendency to effect cognitive functions with psychoorganic syndrome. In addition, good drug tolerability was confirmed. No drug-related adverse events were registered.

### Example 5

The preclinical research studied the ultra low doses (ULD) of activated—potentiated forms of polyclonal affinity purified rabbit antibodies to brain-specific protein S-100 (anti-S100) purified on antigen, and endothelial NO-synthase (anti-eNOS), obtained by super-dilution of initial matrix solution (concentration: 2.5 mg/ml) (100¹², 100³⁰, 100²⁰⁰ times), equivalent to a mixture of centesimal homeopathic dilutions C12, C30, C200 (ratio: 1:1) (ULD anti-S100+anti-eNOS) in treating ischemic stroke caused by prefrontal cerebrocortical photothrombosis in rats.

Acute cerebrovascular disease (brain stroke) ranks third among lethality causes in developed countries and one of the main causes of disability in humans (Gusev E. I., 2003; Janardhan V., Qureshi A. I., 2004).

The photo-induced thrombosis model meets almost all requirements to the experimental model of focal cerebral ischemia. The method developed by Watson (Watson B. et al., 1985) is based on the effect of light with wavelength 560 nm on photosensitive pigment Bengal rose introduced into the blood flow. Active oxygen forms are created and caused increase in adhesiveness of endothelium cells and platelets, and formation of clots closing vascular lumens. The method of ischemic brain lesion induction by using photo-induced thrombosis is technically simple and to close to clinical forms of ischemic brain stroke. A great advantage of this model is that it is non-invasive, i.e. does not require craniotomy and, therefore, more accurately reproduces clinical picture of cerebral thrombosis.

Thirty seven male Wistar rats (weight: 150-180 g; age: 2-3 months) were included in the study of the activity of ULD anti-S100+anti-eNOS in rats with ischemic stroke caused by prefrontal cerebrocortical photothrombosis. Bilateral focal ischemic injury in prefrontal cerebral cortex in rats was induced using the photochemical thrombosis method by Watson (Watson B. D. et al., 1985) as modified by I. V. Viktorov (Romanova G. A. et al, 1998). Bengal rose (3% solution) was injected in the jugular vein of anesthetized rats (n=37) (anesthesia:chloral hydrate 300 mg/kg, intraperitoneally). Using a fiber optic bundle (3 cm in diameter) the light beam from halogen lamp (24 V, 250 W) was delivered to the skull surface above the frontal cortex of the left and right cerebral hemispheres to induce photothrombosis. Sham-operated rats (n=6) were subject to the same procedure except administration of Bengal rose and exposure to halogen lamp light. The intact group included 6 rats.

Five days before and 9 days after stoke induction the following preparations were administered to rats with photothrombosis: distilled water (control-photothrombosis, 5 ml/kg daily, n=12), ULD anti-S100 (5 ml/kg daily, n=7) or ULD anti-S100+anti-eNOS (5 ml/kg daily, n=6). On Day 8 after the operation (or sham operation) conditioned passive avoidance reflex (CPAR) test was performed to assess learning capability and memory in rats. Rats were placed in a unit consisting of illuminated site and connected dark chamber, where animals were exposed to electric foot-shock of 0.45 mA due to which usually preferred dark chamber became dangerous. Development of conditioned passive avoidance reflex was tested on the next day. At that, rats were placed in the illuminated chamber. Latent period of the first entry in the dark chamber was recorded. If a rat avoided the dark chamber for a long time, a conclusion was made that it remembered the danger (electric shock). The longer the latent period of entry in the dark chamber, the better the memory.

Volume of the stroke lesion was morphologically assessed in a proportion of rats of experimental groups on Day 9.

In control rats photothrombosis caused formation of a large stroke area and, therefore, leaded to memory impairment: CPAR reproduction worsened by 9.6% compared to intact rats and by 22.9% compared to sham-operated (Table 5). Administration of ULD anti-S100 reduced the stroke volume by 42.2% and improved memory by 14.0% compared to control-photothrombosis group. Administration of ULD anti-S100+anti-eNOS was more effective: the stroke volume reduced by 44.0%, and conditioned reflex reproduction—by 33.4% compared to control-photothrombosis group.

Therefore, administration of the complex preparation of ULD anti-S100+anti-eNOS was more efficient than monocomponent preparation of ULD anti-S100.

TABLE 5

| | Volume of focal stroke | Latent period of CPAR |
|---|---|---|
| | (mm3); the number of | (seconds), the number of |
| | animals | animals |
| Intact | — | 135.8 ± 28.8; n = 6 |
| Sham-operated | — | 159.3 ± 18.7; n = 6 |
| Control- | 3.41 ± 0.5; n = 9 | 122.8 ± 20.9; n = 12 |
| photothrombosis | | |
| Photothrombosis + | 1.97 ± 0.6; n = 4 | 140.0 ± 26.5; n = 7 |
| ULD anti-S100 | | |
| Photothrombosis + | 1.91 ± 0.5; n = 4 | 163.8 ± 16.2; n = 6 |
| ULD anti-S100 + anti- | | |
| eNOS | | |

What is claimed is:

1. A method of treating organic disease of the nervous system, psychoorganic syndrome or encephalopathy, said method comprising administering a combination pharmaceutical composition comprising a) an activated-potentiated form of an antibody to brain-specific protein S-100 and b) activated-potentiated form of antibodies to endothelial NO synthase.

2. The method of claim 1, wherein said organic disease of the nervous system is acute cerebrovascular disease.

3. The method of claim 2, wherein said acute cerebrovascular disease is ischemic type disease.

4. The method of claim 3, wherein said ischemic type disease is stroke.

5. The method of claim 1, wherein said organic disease of the nervous system is Parkinson's disease.

6. The method of claim 1, wherein the activated-potentiated form of an antibody to brain-specific protein S-100 is to the entire bovine brain-specific protein S-100.

7. The method of claim 1, wherein the activated-potentiated form of an antibody to brain-specific protein S-100 is to brain-specific protein S-100 having SEQ ID NO: 9, SEQ ID NO: 10, SEQ ID NO: 11, or SEQ ID NO: 12.

8. The method of claim 1, wherein the activated-potentiated form of an antibody to endothelial NO synthase is to the entire bovine endothelial NO synthase.

9. The method of claim 1, wherein the activated-potentiated form of an antibody to endothelial NO synthase is to the entire human endothelial NO synthase.

10. The method of claim 1, wherein the activated-potentiated form of an antibody to brain-specific protein S-100 is in the form of a mixture of C12, C30, and C50 homeopathic dilutions impregnated onto a solid carrier and the activated-potentiated form of an antibody to endothelial NO synthase is in the form of mixture of C12, C30, and C200 homeopathic dilutions impregnated onto the solid carrier.

11. The method of claim 1, wherein the activated-potentiated form of an antibody to endothelial NO synthase is in the form of mixture of C12, C30, and C50 homeopathic dilutions impregnated onto a solid carrier and the activated-potentiated form of an antibody to brain-specific protein S-100 is in the form of mixture of C12, C30, and C200 homeopathic dilutions impregnated onto the solid carrier.

12. The method of claim 1, wherein the activated-potentiated form of an antibody to a) brain-specific protein S-100 and b) endothelial NO synthase is a monoclonal, polyclonal or natural antibody.

13. The method of claim 12, wherein the activated-potentiated form of an antibody to a) brain-specific protein S-100 and b) endothelial NO synthase is a polyclonal antibody.

14. The method of claim 1, wherein the activated-potentiated form of an antibody to a) brain-specific protein S-100 and b) endothelial NO synthase is prepared by successive centesimal dilutions coupled with shaking of every dilution.

15. The method of claim 1, wherein the combination pharmaceutical composition is administered in one to two unit dosage forms, each of the dosage form being administered from once daily to six times daily.

16. The method of claim 15, wherein the combination pharmaceutical composition is administered in one to two unit dosage forms, each of the dosage form being administered twice daily.

17. A pharmaceutical composition for use in treating a patient suffering from organic disease of the nervous system, psychoorganic syndrome or encephalopathy, said composition having been obtained by providing a) an activated-potentiated form of an antibody to brain-specific protein S-100 and b) activated-potentiated form of antibodies to endothelial. NO synthase, each prepared by consecutive repeated dilution and multiple shaking of each obtained solution in accordance with homeopathic technology, and then either combining the potentiated solutions by mixing them, or, alternatively, impregnating a carrier mass with said combined solution or with the solutions separately.


SEQUENCE LISTING (12 sequences)

SEQ ID NO: 1 - protein, 1205 aa, Bos taurus
  SOURCE 1..1205: /mol_type=“protein” /organism=“Bos taurus”
MGNLKSVGQEPGPPCGLGLGLGLGLCGKQGPASPAPEPSRAPAPATPHAPDHSPAPNSPTLTRPPEGPKFPRVKNWELGSITYDTLCAQSQQDGPCTPRCCLGSLVLPRKLQTRPSPGPPPAEQLLSQARDFINQYYSSIKRSGSQAHEERLQEVEAEVASTGTYHLRESELVFGAKQAWRNAPRCVGRIQWGKLQVFDARDCSSAQEMFTYICNHIKYATNRGNLRSAITVFPQRAPGRGDFRIWNSQLVRYAGYRQQDGSVRGDPANVEITELCIQHGWTPGNGRFDVLPLLLQAPDEAPELFVLPPELVLEVPLEHPTLEWFAALGLRWYALPAVSNMLLEIGGLEFSAAPFSGWYMSTEIGTRNLCDPHRYNILEDVAVCMDLDTRTTSSLWKDKAAVEINLAVLHSFQLAKVTIVDHHAATVSFMKHLDNEQKARGGCPADWAWIVPPISGSLTPVFHQEMVNYILSPAFRYQPDPWKGSATKGAGITRKKTFKEVANAVKISASLMGTLMAKRVKATILYASETGRAQSYAQQLGRLFRKAFDPRVLCMDEYDVVSLEHEALVLVVTSTFGNGDPPENGESFAAALMEMSGPYNSSPRPEQHKSYKIRFNSVSCSDPLVSSWRRKRKESSNTDSAGALGTLRFCVFGLGSRAYPHFCAFARAVDTRLEELGGERLLQLGQGDELCGQEEAFRGWAKAAFQASCETFCVGEEAKAAAQDIFSPKRSWKRQRYRLSTQAEGLQLLPGLIHVHRRKMFQATVLSVENLQSSKSTRATILVRLDTAGQEGLQYQPGDHIGICPPNRPGLVEALLSRVEDPPPPTESVAVEQLEKGSPGGPPPSWVRDPRLPPCTLRQALTFFLDITSPPSPRLLRLLSTLAEEPSEQQELETLSQDPRRYEEWKWFRCPTLLEVLEQFPSVALPAPLLLTQLPLLQPRYYSVSSAPNAHPGEVHLTVAVLAYRTQDGLGPLHYGVCSTWLSQLKTGDPVPCFIRGAPSFRLPPDPYVPCILVGPGTGIAPFRGFWQERLHDIESKGLQPAPMTLVFGCRCSQLDHLYRDEVQDAQERGVFGRVLTAFSREPDSPKTYVQDILRTELAAEVHRVLCLERGHMFVCGDVTMATSVLQTVQRILATEGDMELDEAGDVIGVLRDQQRYHEDIFGLTLRTQEVTSRIRTQSFSLQERHLRGAVPWAFDPPGPDTPGP

SEQ ID NO: 2 - protein, 1203 aa, Homo sapiens
  SOURCE 1..1203: /mol_type=“protein” /organism=“Homo sapiens”
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 3 - protein, 4 aa, Bos taurus
  SOURCE 1..4: /mol_type=“protein” /organism=“Bos taurus”
PWAF

SEQ ID NO: 4 - protein, 4 aa, Bos taurus
  SOURCE 1..4: /mol_type=“protein” /organism=“Bos taurus”
GAVP

SEQ ID NO: 5 - protein, 21 aa, Bos taurus
  SOURCE 1..21: /mol_type=“protein” /organism=“Bos taurus”
RHLRGAVPWAFDPPGPDTPGP

SEQ ID NO: 6 - protein, 12 aa, Bos taurus
  SOURCE 1..12: /mol_type=“protein” /organism=“Bos taurus”
AFDPPGPDTPGP

SEQ ID NO: 7 - protein, 11 aa, Bos taurus
  SOURCE 1..11: /mol_type=“protein” /organism=“Bos taurus”
HLRGAVPWAFD

SEQ ID NO: 8 - protein, 20 aa, Bos taurus
  SOURCE 1..20: /mol_type=“protein” /organism=“Bos taurus”
HLRGAVPWAFDPPGPDTPGP

SEQ ID NO: 9 - protein, 92 aa, Bos taurus
  SOURCE 1..92: /mol_type=“protein” /organism=“Bos taurus”
MSELEKAVVALIDVFHQYSGREGDKHKLKKSELKELINNELSHFLEEIKEQEVVDKVMETLDSDGDGECDFQEFMAFVAMITTACHEFFEHE

SEQ ID NO: 10 - protein, 92 aa, Homo sapiens
  SOURCE 1..92: /mol_type=“protein” /organism=“Homo sapiens”
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 11 - protein, 94 aa, Bos taurus
  SOURCE 1..94: /mol_type=“protein” /organism=“Bos taurus”
MGSELETAMETLINVFHAHSGKEGDKYKLSKKELKELLQTELSGFLDAQKDVDAVDKVMKELDENGDGEVDFQEYVVLVAALTVACNNFFWENS

SEQ ID NO: 12 - protein, 94 aa, Bos taurus
  SOURCE 1..94: /mol_type=“protein” /organism=“Bos taurus”
MGSELETAMETLINVFHAHSGKEGDKYKLSKKELKELLQTELSGFLDAQKDADAVDKVMKELDENGDGEVDFQEYVVLVAALTVACNNFFWENS